(19) United States
(12) Patent Application Publication
(10) Pub. No.: US 2021/0121502 A1
(43) Pub. Date: Apr. 29, 2021

(54) USE OF PHARMACEUTICAL COMPOSITION IN PREPARING DRUG AGAINST HELICOBACTER PYLORI

(71) Applicants: Li LI, Arcadia, CA (US); BEIJING RONGXIANG INSTITUTE OF REGENERATIVE MEDICINE CO., LTD., Beijing (CN)

(72) Inventor: Li LI, Arcadia, CA (US)

(73) Assignees: BEIJING RONGXIANG INSTITUTE OF REGENERATIVE MEDICINE CO., LTD., Beijing (CN); Li LI, Arcadia, CA (US)

(21) Appl. No.: 16/617,458

(22) PCT Filed: Jun. 4, 2018
(86) PCT No.: PCT/CN2018/089762

§ 371 Date: Nov. 26, 2019

(30) Foreign Application Priority Data
Jun. 6, 2017 (CN) 201710417831.2

(51) Int. Cl. A61K 35/644; A61K 9/00; A61K 47/44; A61K 31/575; A61K 36/539; A61K 36/756; A61K 36/718; A61K 31/7048; A61K 31/366; A61K 31/4375; A61K 31/4741; A61K 35/62; A61K 36/66; A61K 31/565; A61P 31/04

(52) U.S. Cl. CPC A61K 35/644; A61K 2236/35; A61K 47/44; A61K 31/575; A61K 36/539; A61K 36/756; A61K 36/718; A61K 31/7048; A61K 31/366; A61K 31/4375; A61K 31/4741; A61K 35/62; A61K 36/66; A61K 31/565; A61P 31/04; A61K 9/0053

(57) ABSTRACT

A use of a pharmaceutical composition in preparing a drug against Helicobacter pylori. The pharmaceutical composition is suitable for oral administration and comprises a homogeneous mixture of edible oil, beeswax, and β-sitosterol. The beeswax in the composition forms microcrystals. Based on the total weight of the composition, the content of the beeswax is 0.5-50%, and the content of the β-sitosterol is 0.1-20%. The pharmaceutical composition can be used in inhibiting or killing Helicobacter pylori and in treating or preventing a disease caused by Helicobacter pylori.

### FIELD OF THE INVENTION

The present invention relates to a use of a pharmaceutical composition in the preparation of medicaments of anti-Helicobacter pylori. The present invention also relates to a use of a pharmaceutical composition in the preparation of a medicament for the treatment/prevention of diseases caused by Helicobacter pylori.

### BACKGROUND OF THE INVENTION

Chinese patent ZL 02105541.6 discloses a pharmaceutical composition suitable for oral administration, comprising a homogenous mixture of edible oil, beeswax and β-sitosterol, wherein the beeswax in the composition forms microcrystals, the content of the beeswax is 0.5 to 50% and the content of the β-sitosterol is at least 0.1% by weight based on the total weight of the composition. In addition, the composition can also comprise other pharmaceutical ingredients, and is used to deliver other active ingredients to the gastrointestinal tract for treating various diseases.

Moreover, this pharmaceutical composition is mainly used to protect mucosal tissues from damage caused by irritants, and to promote the repair and regeneration of damaged or incomplete gastrointestinal mucosal tissues. It is particularly used for the treatment of gastrointestinal disorders such as gastritis, peptic ulcer, reflux esophagitis, dyspepsia and gastric cancer, as well as for the reconstruction of the physiological structure and function of mucosal tissues.

In the present application, “pharmaceutical composition”, “pharmaceutical composition according to the present invention” or “the present pharmaceutical composition” refers to a pharmaceutical composition comprising a homogenous mixture of edible oil, beeswax and β-sitosterol, wherein the beeswax in the composition forms microcrystals, the content of the beeswax is 0.5 to 50% and the content of the β-sitosterol is 0.1 to 20% by weight based on the total weight of the composition.

Helicobacter pylori (HP) is a spiral or sigmoid, microaerophilic Gram-negative bacterium that exclusively settles in human stomach. It is the main cause of human acute and chronic gastritis, peptic ulcer (gastric ulcer and duodenal ulcer), gastric cancer, gastric non-Hodgkin's lymphoma and gastric mucosa-associated lymphoid tissue (MALT) lymphoma. HP infection rate is as high as 40 to 90% in human. HP is usually infected in childhood. Once infected, the carrier will carry HP for life, and become the infection source of HP.

People are usually infected in childhood, the infection rate of people under 5 years old reaches 50%. This bacterial infection first causes chronic gastritis, and leads to gastric ulcer and gastric atrophy, and in severe cases it develops into gastric cancer. According to statistics, people who are initially infected with HP at an early age have a high incidence of atrophic gastritis and gastric cancer. HP infection has a parallel relationship with the mortality rate of gastric cancer. HP parasitizes in gastric mucosa. 67% to 80% of gastric ulcers and 95% of duodenal ulcers are caused by HP. The common symptom of patients with chronic gastritis and peptic ulcer is fullness, discomfort or pain in the upper abdomen after eating, which is often accompanied by other adverse symptoms such as belching, bloating, sour regurgitation and loss of appetite. Some patients may also have recurrent severe abdominal pain, a small amount of bleeding in the upper gastrointestinal tract and the like.

The continuous discovery of effective drugs has become a top priority due to the strong pathogenicity of HP in the digestive system.

In the prior art, the drug treatment of HP usually uses antibacterial drugs such as omeprazole, amoxicillin, metronidazole tablets and the like. Currently in the world, antibiotics for clinical use against HP are expensive and prone to drug resistance, and have obvious toxic and side effects. The overall efficacy is thereof not satisfactory.

### SUMMARY OF THE INVENTION

The technical problem to be solved by the present invention is to inhibit or kill HP by using the above known pharmaceutical composition, thereby treating diseases caused by HP.

In an aspect, the present invention thus relates to a use of a pharmaceutical composition in the preparation of medicaments of anti-HP. The pharmaceutical composition is a pharmaceutical composition suitable for oral administration comprising a homogenous mixture of edible oil, beeswax and β-sitosterol, wherein the beeswax in the composition forms microcrystals, the content of the beeswax is 0.5 to 50% and the content of the β-sitosterol is 0.1% to 20% by weight based on the total weight of the composition.

Specifically, “anti-HP” intends to make HP unable to grow and reproduction, slow down HP reproduction, or make HP variation and death, or reduce its pathogenicity.

In a specific embodiment, “to make HP unable to grow and reproduction” means that the pharmaceutical composition of the present invention is capable of directly killing HP, and HP is completely unable to grow and reproduce. “to slow down HP reproduction” means that the pharmaceutical composition of the present invention can allow HP to reproduce at certain degree, but the reproduction is limited, followed by morphological mutation that is a transition stage before death, and the bacteria eventually die.

In a specific embodiment, “to reduce its phthogenicity” means that the pharmaceutical composition of the present invention is capable of inhibiting the killing effect of HP on cells, i.e., reducing its toxicity.

The effect of normal cultured HP on cells is significantly killing. However, the effect of cultured HP on cells after the addition of the pharmaceutical composition can be divided into different situations: the higher the concentration of the pharmaceutical composition, the stronger the inhibition of the bacteria and the smaller the effect on cell growth; the lower the concentration of the pharmaceutical composition, the weaker the inhibition of the bacteria, the greater the effect on cell growth, and the greater the killing effect on the cells.

In another aspect, the present invention relates to a use of a pharmaceutical composition in the preparation of medicaments for the treatment or prevention of diseases caused by HP, wherein the pharmaceutical composition is a pharmaceutical composition suitable for oral administration comprising a homogenous mixture of edible oil, beeswax and β-sitosterol, wherein the beeswax in the composition forms microcrystals, the content of the beeswax is 0.5 to 50% and the content of the β-sitosterol is 0.1% to 20% by weight based on the total weight of the composition.

Specifically, the disease caused by HP comprises gastritis, gastric ulcer, duodenal ulcer, gastric cancer, gastric non-Hodgkin's lymphoma and gastric mucosa-associated lymphoid tissue lymphoma caused by HP infection.

Specifically, the disease caused by HP is a disease caused in a mammal, preferably a human.

In a specific embodiment, the content of the β-sitosterol in the pharmaceutical composition is 0.5 to 20% by weight.

In a specific embodiment, the content of the β-sitosterol in the pharmaceutical composition is 1 to 10% by weight.

In a specific embodiment, the content of the beeswax in the pharmaceutical composition is 3 to 30% by weight.

In a specific embodiment, the content of the beeswax in the pharmaceutical composition is 5 to 20% by weight.

In a specific embodiment, the content of the beeswax in the pharmaceutical composition is 6 to 10% by weight.

In a specific embodiment, the edible oil in the pharmaceutical composition is corn oil, wheat germ oil, soybean oil, rice bran oil, rapeseed oil, sesame oil or fish oil.

In a specific embodiment, the pharmaceutical composition further comprises propolis, and the content thereof is 0.1 to 30% by weight.

In a specific embodiment, the pharmaceutical composition comprises water, and the content thereof is less than or equal to 1% by weight.

In a specific embodiment, the dosage form of the oral pharmaceutical composition is selected from the group consisting of a tablet, pill, capsule, emulsion, gel, syrup and suspension.

In a specific embodiment, the pharmaceutical composition further comprises Scutellaria baicalensis or the extract of Scutellaria baicalensis, and the content of Scutellaria baicalensis or the extract of Scutellaria baicalensis (having 0.1 to 0.5% of baicalin) is 2 to 5% by weight based on the total weight of the composition.

The extract of Scutellaria baicalensis is an extract of Scutellaria baicalensis with water, organic solvent such as oil and ethanol, or a combination of water and organic solvent. More preferably, the extract is an extract of 1 to 50% by weight of Scutellaria baicalensis in an edible oil, preferably sesame oil. The radix of Scutellaria baicalensis is preferred. Scutellaria baicalensis is one or more Labiatae plants selected from the group consisting of Scutellaria viscidula bunge, Scutellaria amoena, Scutellaria rehderiana Diels, Scutellaria ikonnikovii Juz, Scutellaria likiangensis and Scutellaria hypericifolia.

In a specific embodiment, the pharmaceutical composition further comprises Cortex phellodendri or or the extract of Cortex phellodendri, and the content of Cortex phellodendri or the extract of Cortex phellodendri (having 0.1 to 1% of obaculactone) is 2 to 5% by weight based on the total weight of the composition.

The the extract of Cortex phellodendri is an extract of Cortex phellodendri with water, organic solvent such as oil and ethanol, or a combination of water and organic solvent. More preferably, the extract is an extract of 1 to 50% by weight of Cortex phellodendri in an edible oil, preferably sesame oil. The cortex of Cortex phellodendri is preferred. Cortex phellodendri is one or more plants selected from the group consisting of Phellodendron chinense Schneid, Phellodendron amurense, Phellodendron chinense Schneid var. omeiense, Phellodendron Schneid var. yunnanense and Phellodendron chinense Schneid var. falcutum.

In a specific embodiment, the pharmaceutical composition further comprises 2 to 5% of Coptis chinensis or the extract of Coptis chinensis (having 0.1 to 1% of berberine) by weight based on the total weight of the composition.

The extract of Coptis chinensis is an extract of Coptis chinensis with water, organic solvent such as oil and ethanol, or a combination of water and organic solvent. Preferably, the extract is an extract of 1 to 50% by weight of Coptis chinensis in an edible oil, preferably sesame oil. The radix of Coptis chinensis is preferred. Coptis chinensis is one or more Ranunculaceae plants selected from the group consisting of Coptis deltoidea C. Y. Cheng et Hsial, Coptis omeiensis and Coptis teeta Wall.

In a specific embodiment, the pharmaceutical composition further comprises 2 to 5% of Scutellaria baicalensis or the extract of Scutellaria baicalensis (having 0.1 to 0.5% of baicalin), 2 to 5% of Cortex phellodendri or the extract of Cortex phellodendri (having 0.1 to 1% of obaculactone), 2 to 5% of Coptis chinensis or the extract of Coptis chinensis (having 0.1 to 1% of berberine), 2 to 10% of Pericarpium papaveris or the extract of Pericarpium papaveris (having 0.1 to 1% of narcotoline), and 2 to 10% of earthworm or earthworm extract containing amino acid, by weight based on the total weight of the composition.

The extract of Pericarpium papaveris is an extract of Pericarpium papaveris with water, organic solvent such as oil and ethanol, or a combination of water and organic solvent. Preferably, the extract is an extract of 1 to 50% by weight of Pericarpium papaveris in an edible oil, preferably sesame oil.

The earthworm extract is an extract of earthworm with water, organic solvent such as oil and ethanol, or a combination of water and organic solvent. More preferably, the extract is an extract of 1 to 50% by weight of earthworm in an edible oil.

The extraction of Scutellaria baicalensis, Cortex phellodendri, Coptis chinensis, Pericarpium papaveris and earthworm can be carried out according to the method described in Chinese Patent ZL 93100276.1 or Chinese Patent ZL 02105541.6.

In a specific embodiment, the pharmaceutical composition comprises 7% of beeswax, 1% of sterol, 0.5% of obaculactone, 0.3% of baicalin and 0.5% of berberine by weight based on the total weight of the composition.

In a specific embodiment, the beeswax has microcrystals with a length of 0.1 to 100 microns.

In a specific embodiment, at least two microcrystals of the beeswax in the pharmaceutical composition are polymerized into a microcrystal complex.

In a specific embodiment, the microcrystals of the beeswax are sufficiently uniformly dispersed in the edible oil.

The clinical application value of the pharmaceutical composition of the present invention is that the pharmaceutical composition of the present invention strongly inhibits the growth of HP and has a strong antibacterial effect on HP, indicating the direction for future research and development. The results of the present invention demonstrate that the pharmaceutical combination of the present invention is an excellent “antibiotic” against HP, and can be used for treating diseases such as gastritis, gastric ulcer, duodenal ulcer, gastric cancer and gastric mucosa-associated lymphoid tissue lymphoma.

### DESCRIPTION OF THE DRAWINGS

FIG. 1A: HP cultured in Columbia medium in Example 2, which shows normal growth HP with normal morphology dyeing (DIC, ×1000).

FIG. 1B: In Example 2, no HP is survived after 72 hours of culture in Columbia medium containing 20% of the present pharmaceutical composition (DIC, ×1000).

FIG. 1C: In Example 2, no HP is survived after 72 hours of culture in Columbia medium containing 5% of the present pharmaceutical composition (DIC, ×1000).

FIG. 2A: In Example 2, HP shows normal morphology and has no variation after 3 days of culture in Columbia medium containing 1.25% of the present pharmaceutical composition (DIC, ×1000).

FIG. 2B: In Example 2, after 5 days of culture in Columbia medium containing 1.25% of the present pharmaceutical composition, HP has mutated, mainly as the cell bodies become longer (DIC, ×1000).

FIG. 2C: In Example 2, after 7 days of culture in Columbia medium containing 1.25% of the present pharmaceutical composition, HP has much more and obvious variation, mainly as the cell bodies become much longer and the death of the variant bacteria increases. The background is the dead HP (DIC, ×1000).

FIG. 2D: In Example 2, after 9 days of culture in Columbia medium containing 1.25% of the present pharmaceutical composition, living HP becomes less and less while the death of the variant bacteria is markedly increasing. The background is the dead HP (DIC, ×1000).

FIG. 3A: In Example 3, after 4 days of co-cultivation, no OMEC is observed in 3 ml of normal HP suspension under the microscope (DIC, ×600).

FIG. 3B: In Example 3, after 4 days of co-cultivation, OMEC could be observed in 3 ml of variant HP suspension under the microscope (DIC, ×600).

FIG. 4A: In Example 4, the results of co-cultivation of HP and OEMC for 17 days in Columbia medium free of the present pharmaceutical composition (DIC, ×600).

FIG. 4B: In Example 4, the results of co-cultivation of HP and OEMC for 17 days in Columbia medium containing 0.3125% of the present pharmaceutical composition (DIC, ×600).

FIG. 4C: In Example 4, the results of co-cultivation of HP and OEMC for 17 days in Columbia medium containing 1.25% of the present pharmaceutical composition (DIC, ×600).

FIG. 4D: In Example 4, the results of the normal control group after 17 days of co-cultivation (DIC, ×600).

FIG. 5A: In Example 5, OMEC are not completely dead, and typical morphology could still be observed on Day 46 of the co-cultivation of OMEC with variant HP suspension (DIC, ×600).

FIG. 5B: In Example 5, OMEC still grows well, with a typical morphology on Day 46 for the normal control (DIC, ×600).

FIG. 6: In Example 6, the stereomicroscope inspection reveals that there is not any bacterial grows in the Columbia medium (stereomicroscope, ×8).

FIG. 7A: In Example 8, HP cultured in Columbia medium grows well with the normal morphology (DIC, ×1000).

FIG. 7B: In Example 8, 72 hours of cultivation of HP in Columbia medium containing 1.25% of the present pharmaceutical composition, there is extremely little bacterial growth in the medium (DIC, ×1000).

FIG. 8A: In Example 8, HP cultured in Columbia medium grows well with the normal morphology (DIC, ×1000).

FIG. 8B: In Example 8, HP cultured in Columbia medium containing 1.25% of the present pharmaceutical composition is in the early stage of variation, mainly manifesting as the cell body becomes longer (DIC, ×1000).

FIG. 8C: In Example 8, HP cultured in Columbia medium containing 1.25% of the present pharmaceutical composition is in the later stage of variation, mainly manifesting as the cell body becomes thinner and longer. The figure shows the dead HP (DIC, ×1000).

### DETAILED DESCRIPTION OF THE INVENTION

The present invention is further illustrated by the following examples in combination with the figures, which should not be construed as a limitation to the present invention. Specific materials and sources thereof used in the embodiments of the present invention are provided below. However, it should be understood that these are merely exemplary and are not intended to limit the present invention. Materials that are 25 the same as or similar to the following reagents and instruments in type, model, quality, properties or function can also be used in the embodiment of the present invention. Unless otherwise specified, the experimental methods used in the following examples are conventional methods, and the materials, reagents and the like used in the following examples are commercially available.

### Example 1: Preparation of the Present Pharmaceutical Composition

The pharmaceutical composition was prepared according to the method disclosed in Example 1 of Chinese Patent ZL 02105541.6.

Briefly, step 1: the refined sesame oil and Scutellaria baicalensis (100 kg:5 kg) were added to a reaction tank and heated. Heating was stopped when the temperature reached 120° C., and the mixture was kept warm for 50 minutes with stirring. The mixture was filtrated to remove the dregs, the obtained extraction was the medicinal oil I.

Step 2: the medicinal oil I was added to another reaction tank and heated. When the temperature reached 85° C., the refined beeswax was added following a ratio of 193 kg of medicinal oil:7 kg of beeswax, and stirred well. Stop heating when the temperature reached 120° C., kept stirring the warm mixture for 20 minutes, then, the medicinal oil II was ready.

Step 3: the medicinal oil II was grinded using a colloid mill with a pitch of 0.6 to 0.8 mm and an output speed of 15 Kg/15 min. Alternatively, the medicinal oil II could also be homogenized at 40±2° C. for 15 to 20 minutes using a homogenizer with a rotate speed of 6000 to 10000 rpm. The homogenate was stirred at 100 rpm, vacuumized to below 0.09 MP, cooled to 40±2° C., and kept warm for 50 minutes. When the temperature decreased to 20° C. and the vacuum degree reached 0.6 to 0.8 MP, the mixture was kept for 20 minutes to obtain the pharmaceutical composition.

According to Example 2 of Chinese Patent ZL 02105541.6, the active ingredients of the pharmaceutical composition prepared by the above method are shown in Table 1:

TABLE 1

| Ingredients | Content per 100 g |
|---|---|
| Natural vitamin E | 15 mg~50 mg |
| Total flavone | 20 mg~60 mg |
| β-sitosterol | 0.20 g~1.0 g |
| Linoleic acid | 35 g~55 g |
| Oleic acid | 25 g~45 g |

### Example 2: The Present Pharmaceutical Composition Inhibits HP Growth and Leds to the Variation

1. Materials and Methods

1.1 Instruments, Devices, Materials and Reagents

Ultrapure water system (Milli-Q, Millipore, USA); two-stage reverse osmosis purified water system (Beijing Innogreen Technology Co., Ltd.); electronic scale (AUW220D, Shimadzu, Japan); electronic scale (SCOUT SL SPN402F, authorized by Ohaus, Mettler-Toledo (Changzhou) Weighing Equipment System Co., Ltd.); electronic scale (AB135-S, Mettler-Toledo, Switzerland); electronic scale (ES-1000HA, Changsha Xiangping Technology Development Co., Ltd.); floor-standing high-speed refrigerated centrifuge (J20-XP, Beckman-Coulter, USA); desktop high speed refrigerated centrifuge (1-15K, Sigma, Germany); desktop high speed centrifuge (1-14, Sigma, Germany); ultra-low temperature refrigerator (Forma925, Thermo, USA); triple-gas incubator (CB150, Binder, Germany); hybridization oven (Maxi14, Thermo, USA); particle ice machine (SIM-F124, Sanyo, Japan); electronic constant temperature water bath (CS501-3C type), drying oven (Chongqing Sida Experimental Instrument Co., Ltd.); inverted microscope (TE2000U, Nikon, Japan); upright microscope (E800, Nikon, Japan); microscopic imaging system (DXM 1200, Nikon, Japan); ordinary inverted microscope (XDS-1B), ordinary optical microscope (BK1201) (Chongqing Optical Instrument Factory); biological clean bench (BCN-1360B), biological safety cabinet (BSC-IIA2), biochemical incubator (HPS-200B) (Beijing HDL Instrument Manufacturing Co., Ltd.); Helicobacter pylori (ATCC43504, Shanghai Beisi Biotechnology Co., Ltd.); Columbia blood agar base (CBAB, CM0331, OXOID Co., UK); brain heart extract (BHI, CM1135, OXOID Co., UK); nutrient agar medium (NAM, Beijing Sanyao Technology Development Co., Ltd.); slides and coverslips (Sinopharm Chemical Reagent Beijing Co., Ltd); nutrient agar (NA), Gram stain (crystal violet, iodine solution, 95% ethanol, safranin), large filter paper, sterile defibrated sheep blood, xylene, 3% hydrogen peroxide, N,N,N,N-tetramethyl-p-phenylenediamine dihydrochloride (TMPD), trimethoprim TMP, polymyxin B sulfate, soluble amphotericin B, vancomycin hydrochloride, DL-lactic acid, triangular glass spreading rod (Beijing Solarbio Technology Co., Ltd.); one-minute rapid Helicobacter pylori test paper (chemical reaction method) (Zhuhai Kedi Technology Co., Ltd.); disposable culture dish (apius, Qingdao Jindian Biochemical Equipment Co., Ltd.); various types of syringes; 6-well culture plate (Costar, US); 50 ml centrifuge tube; Eppendorf centrifuge tube; micro-sampler (1000 μl, 200 μl, 20 μl, 10 μl, Gilson, France); dripper; culture dish (φ5 cm, (φ6 cm); 0.22 μm microporous membrane; needle filter; capped triangular flask; capped small test tube; needle; DMEM medium (GIBCO, Invitrogen Corporation, USA); fetal bovine serum (FBS, ExCell Co.); penicillin sodium for injection; streptomycin sulfate for injection; large glass test tube.

1.2 Methods

1.2.1 Preparation of Columbia Medium Containing the Present Pharmaceutical Composition

3.9 g of Columbia blood agar base (CBAB) was added to a clean 250 ml conical flask, added 100 ml of ultrapure water. Heat the mixture on an induction cooker until CBAB was dissolved in boiling water. The conical flask was sealed with a cotton plug, tightened with cord, and autoclaved at 121° C. for 15 minutes. The medium was immediately removed from the autoclave when the pressure decreased to zero, and added certain amount of the pharmaceutical composition by sterile operation. The medium was sealed with a sterile cotton plug, covered with sterile kraft paper, and shaked frequently to dissolve the pharmaceutical composition. The medium was further cooled to about 50° C., added with 8 ml of sterile defibrinated sheep blood, mixed well, and poured rapidly onto the plate when it was still warm. The plate was covered, cooled, marked, placed up side down, and stored at 4° C.

1.2.2 Identification of HP

Colony: The colony on the plate was needle-like, ground-glass-like and moist, with a diameter of 1 to 2 mm. If the amount of inoculated bacteria was large, the colonies would fuse on the surface of the plate to form a layer of translucent lawn.

Morphology: One drop of saline was dropped on the center of a clean slide. An appropriate amount of bacteria was scraped with a sterile ring, placed in the saline and spreaded into a thin film. The film was naturally dried or dried by alcohol burner, and subjected to Gram stain. Gram stain steps: the film was soaked with crystal violet solution for 1 minute, and rinsed with water; then soaked with iodine solution for 1 minute, and rinsed with water; then soaked with 95% ethanol for 30 seconds, and rinsed with water; then soaked with safranin solution for 1 minute, and rinsed with water; and then dried. The bacteria were Gram-negative under the microscope, being spiral, curved or sigmoid prunosus bacilli with various lengths.

Biochemical Reaction

Oxidase reaction: Preparation of the reagent: 0.02606 g of TMPD was dissolved in 2.61 ml of sterile ultrapure water to obtain a 1% TMPD solution, which was stored in the dark at 4° C. During the identification, a strip of filter paper was fixed on a slide, and stained with suspected bacteria by a ring. One drop of the above formulated 1% TMPD solution was added rapidly onto the filter paper, and the positive bacteria would quickly develop a dark blue/black reaction on the bacteria site.

Catalytic reaction: A clean concave slide was stained with suspected bacteria by a ring at the center. One drop of 3% H₂O₂ was added rapidly onto the concave slide, and the positive bacteria would quickly develop continuous oxygen bubbles.

Urease reaction: A HP test strip was stained with suspected bacteria by a ring. The bacteria was spreaded on the HP test strip, and the positive bacteria would immediately develop a bright red color on the spreaded site.

1.2.3 Preservation and Recovery of HP

Preparation of the cryopreservation solution: 1.85 g of brain heart extract (BHI) was added to a clean 150 ml conical flask, and added with 50 ml of ultrapure water. BHI was dissolved in boiling water on induction cooker. The conical flask was sealed with a cotton plug, tightened with cord, and autoclaved at 121° C. for 15 minutes. After the solution was cooled to room temperature, added 5.6 ml of FBS and mixed well. The solution was dispensed into 15 ml centrifuge tubes with 5 ml per tube, and stored at −20° C.

Cryopreservation of the bacteria: 0.5 ml of cryopreservation solution was added to a cryopreservation tube. A large amount of bacteria in the logarithmic growth phase was scraped with a bacteria extraction ring, and removed from the ring into the cryopreservation solution by grinding against the tube wall to break up the bacteria masses. The tube was capped, marked, and placed in a foam box that had been equilibrated at room temperature. The foam box was placed in a −70° C. ultra-low temperature freezer.

Recovery of the bacteria: The bacteria was taken from the −70° C. ultra-low temperature freezer, and melted rapidly in a 37° C. water bath. The tube surface was cleaned and sterilized, and the bacteria solution was mixed well. 30 μl of bacteria solution was added to the center of the Columbia medium plate, and spreaded into a film with a triangular glass spreading rod. The plate was incubated in a triple-gas incubator at 37° C., 10% CO₂, 5% O₂, 85% N₂, and a relative humidity of 98%.

2. Results

2.1 HP cultured in the Columbia medium, a specific medium for the culture of HP, grows normally, and the morphology, staining and biochemical reaction are normal. However, HP cultured in the Columbia medium containing different concentrations of the pharmaceutical composition could not grow at all. HP could not grow even when the pharmaceutical composition is present in the lowest concentration of 5% (w/v, i.e. 5 g of the pharmaceutical composition was added to 100 ml of Columbia medium).

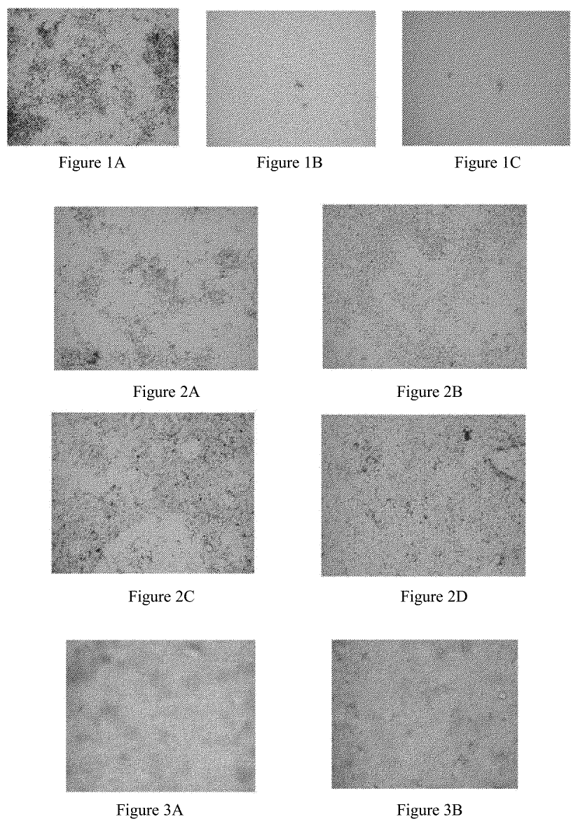

Specifically, HP cultured in Columbia medium grows normally, and the morphology is normal (see FIG. 1A). HP is cultured in a Columbia medium containing 20% (w/v) of the pharmaceutical composition for 72 hours, and the result shows that there is no bacterial growth in the medium (see FIG. 1B). HP is cultured in a Columbia medium containing 10% (w/v) of the pharmaceutical composition for 72 hours, and the result shows that there is no bacterial growth in the medium. HP is cultured in a Columbia medium containing 5% (w/v) of the pharmaceutical composition for 72 hours, and the result shows that there is no bacterial growth in the medium (see FIG. 1C).

2.2 HP cultured in the Columbia medium, a specific medium for the culture of HP, grows normally, and the morphology, staining and biochemical reaction are both normal. However, HP cultured in the Columbia medium containing a low concentration of the pharmaceutical composition has variation obviously in morphology. There is an obvious process of variation, and the variant bacteria all dead eventually.

Specifically, on Day 3 of the culture, HP cultured in the Columbia medium containing 1.25% (w/v) of the pharmaceutical composition is normal in morphology and has no variation (see FIG. 2A). On Day 5 of the culture, HP cultured in the Columbia medium containing 1.25% (w/v) of the pharmaceutical composition shows variation, which mainly manifesting as the cell body becomes longer (see FIG. 2B). On Day 6 of the culture, HP cultured in the Columbia medium containing 1.25% (w/v) of the pharmaceutical composition shows obvious variation, wherein the cell body elongates obviously into a silky shape, and the background is the dead HP. On Day 7 of the culture, HP cultured in the Columbia medium containing 1.25% (w/v) of the pharmaceutical composition shows more obvious variation, which mainly manifesting as a much longer cell body and increased death of the variant bacteria. The background is the dead HP (see FIG. 2C). On Day 8 of the culture, HP cultured in the Columbia medium containing 1.25% (w/v) of the pharmaceutical composition shows even more obvious variation, manifesting as a longer bacterium body with a silky shape, and the variant bacterium decreases. The background is the dead HP. On Day 9 of the culture, HP cultured in the Columbia medium containing 1.25% (w/v) of the pharmaceutical composition is dying obviously, and there is much less alive cells available. The background is the dead HP (see FIG. 2D). On Day 10 of the culture, among HP cultured in the Columbia medium containing 1.25% of the pharmaceutical composition, nearly no alive variant HP left, and the background is a mass of dead HP.

### Example 3: Effect of Normal and Variant HP on Oral Mucosal Epithelial Cells (OMEC)

1. Materials and Methods

1.1 Instruments, Devices, Materials and Reagents

The instruments, devices, materials and reagents are the same as Example 2.

1.2 Methods

1.2.1 Preparation of the Mixed Antibiotics

The Columbia medium contained 10 mg/L vancomycin hydrochloride, 10 mg/L soluble amphotericin B, 2500 U/L polymyxin B sulfate and 5 mg/L trimethoprim. Therefore, 10 mg of vancomycin, 10 mg of soluble amphotericin B, 0.42 mg of polymyxin B sulfate (1 mg=6000 U) and 5 mg of trimethoprim were needed to prepare 1 L of Columbia medium. Since only 100 ml of Columbia medium was prepared everytime, the total amount of each compound was equally divided into 10 portions of the dispensing liquid. The dispensing liquid was 4 ml, which was easy to store, handle and operate. Specific steps were as follows: four sterile 1.5 ml Eppendorf tubes were wrapped with aluminum foil and marked. 10 mg of vancomycin, 10 mg of soluble amphotericin B, 0.42 mg of polymyxin B sulfate and 5 mg of trimethoprim were weighed respectively with an electronic scale. The three water soluble antibiotics vancomycin, amphotericin B and polymyxin B sulfate were added to Eppendorf tubes. Trimethoprim was treated as follows: trimethoprim was added to a sterile large glass test tube, add 10 ml of sterile ultra-pure water to rinse the compound to the tube bottom, then add 20 μl of DL-lactic acid; the tube was clamped with a test tube holder, sealed with a cotton plug, heated with an alcohol burner to boiling for 10 minutes, then cool to room temperature. The other three antibiotics tubes were added with 1 mL of sterile ultra-pure water respectively, capped, and shaked to dissolve the solid. The other three antibiotics were added to a 50 ml centrifuge tube, and the Eppendorf tubes were rinsed with sterile ultra-pure water once to twice. The cooled trimethoprim solution was added to the centrifuge tube, rinsed with ultra-pure water, and added with sterile ultra-pure water until to 40 ml. The 40 ml of mixed antibiotics solution was filtrated through a needle filter into another 50 ml sterile centrifuge tube, and dispensed into ten sterile 15 ml centrifuge tubes (4 ml per tube). The centrifuge tubes were sealed, marked, and stored at 20° C. 100 ml of medium was prepared by dissolving the medium in 96 ml of ultra-pure water, and adding one tube of mixed antibiotics solution (4 ml) before pouring the medium onto the plate.

1.2.2 Preparation of Columbia Medium

3.9 g of Columbia blood agar base (CBAB) was added to a clean 250 ml conical flask that contained 100 ml of ultra-pure water. Then, the flask was heated on an induction cooker until CBAB was dissolved in the boiling water. The conical flask was sealed with a cotton plug, tightened with cord, and autoclaved at 121° C. for 15 minutes. The medium was cooled to about 50° C., added with 8 ml of sterile defibrinated sheep blood, mixed well, and poured rapidly onto the plate when it was still hot. The plate was covered, cooled, marked, placed up side down, and stored at 4° C.

1.2.3 Preparation of Columbia Medium Containing the Mixed Antibiotics

3.9 g of Columbia blood agar base (CBAB) was added to a clean 250 ml conical flask that contained 100 ml of ultra-pure water. Then, the flask was heated on an induction cooker until CBAB was dissolved in the boiling water. The conical flask was sealed with a cotton plug, tightened with cord, and autoclaved at 121° C. for 15 minutes. The medium was cooled to about 50° C., added with 4 ml of mixed antibiotics solution and 8 ml of sterile defibrinated sheep blood, mixed well, and poured rapidly onto the plate when it was still hot. The plate was covered, cooled, marked, placed up side down, and stored at 4° C.

1.2.4 Preparation of Columbia Medium Containing the Pharmaceutical Composition

3.9 g of Columbia blood agar base (CBAB) was added to a clean 250 ml conical flask that contained 100 ml of ultra-pure water. Then, the flask was heated on an induction cooker until CBAB was dissolved in the boiling water. The conical flask was sealed with a cotton plug, tightened with cord, and autoclaved at 121° C. for 15 minutes. The medium was immediately removed from the autoclave when the pressure decreased to zero, and added immediately with a certain amount of the pharmaceutical composition by sterile operation. The medium was sealed with a sterile cotton plug, covered with sterile kraft paper, and shaked frequently to melt and dissolve the pharmaceutical composition. The medium was further cooled to about 50° C., added with 8 ml of sterile defibrinated sheep blood, mixed well, and poured rapidly onto the plate when it was still hot. The plate was covered, cooled, marked, placed up side down, and stored at 4° C.

1.2.5 Identification of HP

The identification of HP was as same as Part 1.2.2 of Example 2.

1.2.6 Preservation and Recovery of HP

The preservation and recovery of HP were as same as Part 1.2.3 of Example 2.

1.2.7 Culture of OMEC and Co-Cultivation with Normal and Variant HP

The buccal OMEC was scraped with a sterile disposable flocking swab, and released in PBS contained the double-antibody in an ice bath;

The cells were counted. The solution was centrifugated (2000 rpm) at 4° C. for 5 minutes, and the supernatant was discarded;

3 ml of pre-cooled 10% FBS DMEM cell culture medium was added, gently vortex to mix the cells well, followed by adding 10 ml of the same DMEM culture medium;

The solution was centrifugated (2000 rpm) at 4° C. for 5 minutes, and the supernatant was discarded;

3 ml of pre-cooled 10% FBS DMEM cell culture medium was added, gently vortex to mix the cells well, followed by adding 10 ml of the same DMEM culture medium;

The above cell suspension was equally divided into each well of a 6-well plate, an appropriate amount of 10% FBS DMEM cell culture solution was added to each well, and the total volume of each well was 5 ml.

The cells were cultured in a cell incubator at 37° C., 5% CO₂;

HP was cultured on 3 days and 5 days in advance to obtain normal HP and variant HP, respectively. On Day 3 of the culture, normal HP cultured in the Columbia medium free of the pharmaceutical composition formed an obvious lawn. A half of the total area of the lawn was scraped by a cell scraper, transferred to 4 ml of 10% FBS DMEM, and gently ground into a uniform normal HP suspension with the dripper. On Day 5 of the culture, HP cultured in the Columbia medium containing 0.3125% (w/v) of the pharmaceutical composition was treated by the same method to obtain 4 ml of a variant HP suspension.

On Day 1 of the OMEC culture, different HP suspensions were added to different wells of the plate as follows:

A1 well: 1 ml of supernatant was taken away, and 3 ml of normal HP suspension 25 was added;

B1 well: no supernatant was taken away, and 1 ml of normal HP suspension was added;

A2 well: 1 ml of supernatant was taken away, and 3 ml of variant HP suspension was added;

B2 well: no supernatant was taken away, and 1 ml of variant HP suspension was added;

Culture medium was added to each well to make up the final volume, and A3 and B3 wells were normal blank control wells;

The plate was incubated continuously in a incubator at 37° C., 5% CO₂, and cell 40 growth, morphology and structure were recorded once or twice a day;

The same position of different wells was used during cell photographing;

The cells were observed with a Nikon TE2000U inverted microscope, and images were recorded with a Nikon DMX1200. The image resolution could be adjusted as needed. The observation time was shortened as much as possible, and the experimental results were properly saved.

2. Results

On Day 3 of the culture, HP normally cultured in the Columbia medium free of the pharmaceutical composition forms an obvious lawn, the colonies are typical, and Gram stain shows that the bacteria has normal morphology, and has no variation. On Day 5 of the culture, HP cultured in the Columbia medium containing 0.3125% (w/v) of the pharmaceutical composition has obvious variation in morphology, according to Gram stain.

The effect of normal and variant HP culture on OMEC growth is observed through co-cultivation of OMEC and HP as follows:

On Day 4 of the co-cultivation, no OMEC is observed in 3 ml (FIG. 3A) and 1 ml of normal HP suspension under the microscope, indicating that all OMEC has been killed by HP culture. On Day 4 of the co-cultivation, OMEC could be observed in 3 ml (FIG. 3B) and 1 ml of variant HP suspension under the microscope, indicating that OMEC has not been killed by HP culture. On Day 4 of the co-cultivation, normal OMEC could be observed in the normal blank control well under the microscope, and are in health situation.

On Day 15 of the co-cultivation, no OMEC is observed in 3 ml and 1 ml of normal HP suspension under the microscope, indicating that OMEC has been killed by HP culture. On Day 15 of the co-cultivation, OMEC could be observed in 3 ml and 1 ml of variant HP suspension under the microscope, indicating that OMEC has not been killed by HP culture. On Day 15 of the co-cultivation, normal OMEC could be observed in the normal blank control well under the microscope, and are in health situation.

On Day 41 of the co-cultivation, no OMEC is observed in 3 ml and 1 ml of normal HP suspension under the microscope, indicating that OMEC has been killed by HP structure. On Day 41 of the co-cultivation, OMEC could be observed in 3 ml and 1 ml of variant HP suspension under the microscope, indicating that OMEC has not been killed by HP culture. On Day 41 of the co-cultivation, normal OMEC could be observed in the normal blank control well under the microscope, and are in health situation.

### Example 4: Effect of the Present Pharmaceutical Composition on the Toxicity of HP Under Culture Conditions In Vitro

1. Materials and Methods

1.1 Instruments, Devices, Materials and Reagents

The instruments, devices, materials and reagents are as same as Example 3.

1.2 Methods

1.2.1 to 1.2.6 are as same as Example 3.

1.2.7 Culture of OMEC and Co-Cultivation with HP Culture

The buccal OMEC was scraped with a sterile disposable flocking swab, and released in PBS contained the double-antibody in an ice bath;

The cells were counted. The solution was centrifugated (2000 rpm) at 4° C. for 5 minutes, and the supernatant was discarded;

3 ml of pre-cooled 10% FBS DMEM cell culture medium was added, gently vortex to mix the cells well, followed by adding 10 ml of the same DMEM culture medium;

The solution was centrifugated (2000 rpm) at 4° C. for 5 minutes, and the supernatant was discarded;

3 ml of pre-cooled 10% FBS DMEM cell culture medium was added, gently vortex to mix the cells well, followed by adding 10 ml of the same DMEM culture medium;

The above cell suspension was equally divided into each well of a 6-well plate, an appropriate amount of 10% FBS DMEM cell culture solution was added to each well, and the total volume of each well was 5 ml.

The cells were cultured in an incubator at 37° C., 5% CO₂;

HP was cultured 3 days in advance in three different medium, wherein medium I was a Columbia medium free of the pharmaceutical composition, medium II was a Columbia medium containing 0.3125% (w/v) of the pharmaceutical composition, and medium III was a Columbia medium containing 1.25% (w/v) of the pharmaceutical composition. On Day 3 of the culture, HP had formed an obvious lawn. The total area or a half of the total area of the lawn was scraped by a cell scraper, transferred to 4 ml of 10% FBS DMEM, and gently ground into a uniform HP suspension with the dripper. Thus three different HP suspensions were obtained.

On Day 3 of the OMEC culture, different HP suspensions were added to different wells of the plate as follows:

A1 well: 2 ml of supernatant was taken away, and 4 ml of HP suspension obtained from medium I was added.

A2 well: 2 ml of supernatant was taken away, and 4 ml of HP suspension obtained from medium II was added.

A3 well: 2 ml of supernatant was taken away, and 4 ml of HP suspension obtained from medium III was added.

B1, B2 and B3 wells were added respectively with 2 ml 10% FBS DMEM, and used as the normal blank control well.

The plate was incubated continuously in a incubator at 37° C., 5% CO₂, and cell growth, morphology and structure were recorded once or twice a day;

The same position of different wells was used during cell photographing;

The cells were observed with a Nikon TE2000U inverted microscope, and images were recorded with a Nikon DMX1200. The image resolution could be adjusted as needed. The observation time was shortened as much as possible, and the experimental results were properly saved.

2. Results

HP were cultured in three different Columbia medium containing different contents of the pharmaceutical composition. On Day 3 of the culture, HP cultured in the Columbia medium free of the pharmaceutical composition (medium I) are Gram-negative, there are a lot of bacteria, and OMEC morphology is normal; HP cultured in the Columbia medium containing 0.3125% (w/v) of the pharmaceutical composition (medium II) are Gram-negative, there are a lot of bacteria, and OMEC morphology is normal; HP cultured in the Columbia medium containing 1.25% (w/v) of the pharmaceutical composition (medium III) are Gram-negative, OMEC morphology is normal, but there are a few bacteria. HP obtained from medium I and II has formed an obvious lawn, and the colonies are normal. During the co-cultivation, the effect of different HP culture obtained from different medium containing different contents of the pharmaceutical composition on OMEC growth is observed. It is found that the HP culture obtained from medium I has an obvious effect on OMEC, indicating a strong toxicity of HP. The HP culture obtained from medium II also has an obvious effect on OMEC, and OMEC dies obviously, indicating a weak effect of the pharmaceutical composition on the reproduction and toxicity of HP, mainly due to the low concentration. The HP culture obtained from medium III does not have an obvious effect on OMEC, indicating that the pharmaceutical composition exerts its efficacy and inhibits the reproduction and toxicity of HP significantly.

1) On Day 2 of the co-cultivation, no OMEC is observed under the microscope in the co-cultivation of HP cultured in the Columbia medium free of the pharmaceutical composition with OMEC, indicating that all OMEC has been killed by HP culture. On Day 2 of the co-cultivation, no OMEC is observed under the microscope in the co-cultivation of HP cultured in the Columbia medium containing 0.3125% (w/v) of the pharmaceutical composition with OMEC, indicating that OMEC has been killed by HP culture. On Day 2 of the co-cultivation, OMEC grows normally under the microscope in the co-cultivation of HP cultured in the Columbia medium containing 1.25% (w/v) of the pharmaceutical composition with OMEC, indicating that the HP culture does not affect OMEC growth.

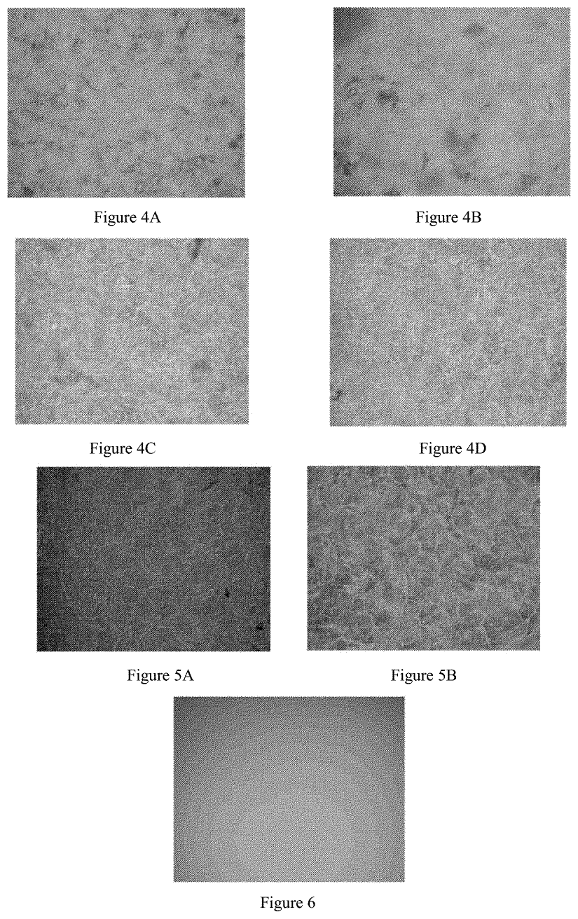

2) On Day 17 of the co-cultivation, no OMEC is observed under the microscope in the co-cultivation of HP cultured in the Columbia medium free of the pharmaceutical composition with OMEC, indicating that the HP culture inhibits OMEC growth significantly (FIG. 4A). On Day 17 of the co-cultivation, no OMEC is observed under the microscope in the co-cultivation of HP cultured in the Columbia medium containing 0.3125% (w/v) of the pharmaceutical composition with OMEC, indicating that the HP culture inhibits OMEC growth significantly (FIG. 4B). On Day 17 of the co-cultivation, in the co-cultivation of HP cultured in the Columbia medium containing 1.25% (w/v) of the pharmaceutical composition with OMEC, OMEC grows normally under the microscope, indicating that the HP culture does not affect OMEC growth (FIG. 4C). On Day 17 of the co-cultivation, OMEC in the normal control group grows normally under the microscope (FIG. 4D).

3) On Day 51 of the co-cultivation, no OMEC growth is observed under the microscope in the co-cultivation of HP cultured in the Columbia medium free of the pharmaceutical composition with OMEC, indicating that the HP culture inhibits OMEC growth significantly. On Day 51 of the co-cultivation, no OMEC growth is observed under the microscope in the co-cultivation of HP cultured in the Columbia medium containing 0.3125% (w/v) of the pharmaceutical composition with OMEC, indicating that the HP culture inhibits OMEC growth significantly. On Day 51 of the co-cultivation, OMEC growth is observed under the microscope in the co-cultivation of HP cultured in the Columbia medium containing 1.25% (w/v) of the pharmaceutical composition with OMEC, indicating that the HP culture does not affect OMEC growth. On Day 51 of the co-cultivation, OMEC in the normal control group grows normally under the microscope.

### Example 5: Reduced Toxicity of the Variant HP

1. Materials and Methods

1.1 Instruments, Devices, Materials and Reagents

The instruments, devices, materials and reagents are as same as Example 3.

1.2 Methods

1.2.1 to 1.2.6 are as same as Example 3.

1.2.7 Culture of OMEC and Co-Cultivation with HP

The buccal OMEC was scraped with a sterile disposable flocking swab, and released in PBS contained the double-antibody in an ice bath;

The cells were counted. The solution was centrifugated (2000 rpm) at 4° C. for 5 25 minutes, and the supernatant was discarded;

3 ml of pre-cooled 10% FBS DMEM cell culture medium was added, vortex to mix the cells well, followed by adding 10 ml of the same DMEM culture medium;

The solution was centrifugated (2000 rpm) at 4° C. for 5 minutes, and the supernatant was discarded;

3 ml of pre-cooled 10% FBS DMEM cell culture medium was added, vortex to mix the cells well, followed by adding 10 ml of the same DMEM culture medium;

The above cell suspension was equally divided into each well of a 6-well plate, an appropriate amount of 10% FBS DMEM cell culture solution was added to each well, and the total volume of each well was 5 ml.

The cells were cultured in an incubator at 37° C., 5% CO₂;

HP was cultured for 6 days in advance in a Columbia medium containing 0.3125% (w/v) of the pharmaceutical composition to obtain the variant HP, which had formed an obvious lawn. A half of the total area of the lawn was scraped by a cell scraper, transferred to 4 ml of 10% FBS DMEM culture solution, and gently ground into 4 ml of a uniform variant HP suspension with the dripper.

On Day 4 of the OMEC culture, 2 ml of culture supernatant was taken away from the well of the plate, and added with the above variant HP culture suspension. The control well was made up with 10% FBS DMEM culture solution.

The plate was incubated continuously in a incubator at 37° C., 5% CO₂, and cell growth, morphology and structure were recorded once or twice a day;

The same position of different wells was used during cell photographing;

The cells were observed with a Nikon TE2000U inverted microscope, and images were recorded with a Nikon DMX1200. The image resolution could be adjusted as needed. The observation time was shortened as much as possible, and the experimental results were properly saved.

2. Results

On Day 6 of the culture, HP cultured in the Columbia medium containing 0.3125% (w/v) of the pharmaceutical composition has variation obviously in morphology according to Gram stain. It was added to the OMEC culture well, and the OMEC growth and the effect of the variant HP culture on OMEC growth were observed.

The results show that the variant HP culture has a certain effect on OMEC, but could not completely kill OMEC, indicating that the toxicity of variant HP has been reduced.

Specifically, on Day 1 of the co-cultivation of OMEC with variant HP suspension, OMEC are not totally dead, a few OMEC with typical morphology could be observed. On Day 20 of the co-cultivation of OMEC with variant HP suspension, OMEC are not all dead, some OMEC with typical morphology could be observed. On Day 24 of the co-cultivation of OMEC with variant HP suspension, OMEC are not dead totally, a lot of OMEC with typical morphology could be observed. On Day 46 of the co-cultivation of OMEC with variant HP suspension, OMEC are not completely dead, OMEC with typical morphology could still be observed (see FIG. 5A). On Day 46 of the normal control, OMEC still grows normally, with a typical morphology (see FIG. 5B).

### Example 6: Effect of DMEM Medium on HP Growth

1. Materials and Methods

1.1 Instruments, Devices, Materials and Reagents

The instruments, devices, materials and reagents are the same as Example 3 in addition to microplate reader (Multiskan Ascent, Labsystems, Finnish), stereomicroscope (SMZ1000, Nikon, Japan) and ELISA plate (Costar, USA).

1.2 Methods

1.2.1 to 1.2.6 are the same as Example 3.

1.2.7 Culture of HP in DMEM Medium

HP was cultured for 3 days in advance in a Columbia medium. On the day of the experiment, the total area of the lawn was scraped by a cell scraper to obtain HP, transferred to 4 ml of 10% FBS DMEM, and gently ground to produce a uniform HP suspension with the dripper. The suspension was vortex, then added 4 ml of 10% FBS DMEM. 200 ul of three test material was added to a detachable ELISA plate and mixed well as follows:

| | |
|---|---|
| 1) HP suspension (HP + DMEM) | 3 parallel wells |
| 2) PBS | 3 parallel wells |
| 3) DMEM | 3 parallel wells |

The OD₆₂₀ value was measured by a Labsystems Multiskan Ascent plate reader at 620 nm wavelength.

Other HP suspensions were mixed well, and divided equally into A1 and A2 wells of the 6-well plate. Each well had about 3 ml of suspension, which was cultured in a cell incubator at 5% O₂, 37° C.

After 36 hours, the HP suspension obtained from the above culture process was cultured in a Columbia medium. 30 ul of the above HP suspension was added to the center of each dish, and spreaded evenly with a triangular glass spreading rod. The cells were cultured in a triple-gas incubator at 10% CO₂, 5% O₂, 85% N₂, 37° C., and HP growth was observed after 5 days. At the same time, the OD₆₂₀ value was measured for the second time by the same method.

After 72 hours, the OD₆₂₀ value was measured for the third time by the same method.

2. Results

At the beginning of the experiment, the OD₆₂₀ value was measured for the first time. The results are as follows:

| | |
|---|---|
| 1) HP + DMEM | 3 wells: 0.484; 0.463; 0.473 |
| 2) PBS | 3 wells: 0.036; 0.037; 0.037; |
| 3) DMEM | 3 wells: 0.081; 0.066; 0.062 |

After 36 hours, the OD₆₂₀ value was measured for the second time. The results are as follows:

| | |
|---|---|
| 1) HP + DMEM | 3 wells: 0.325; 0.350; 0.301 |
| 2) PBS | 3 wells: 0.035; 0.035; 0.036; |
| 3) DMEM | 3 wells: 0.060; 0.064; 0.068 |

After 72 hours, the OD₆₂₀ value was measured for the third time by the same method. The results are as follows:

| | |
|---|---|
| 1) HP + DMEM | 3 wells: 0.284; 0.271; 0.267 |
| 2) PBS | 3 wells: 0.035; 0.035; 0.036; |
| 3) DMEM | 3 wells: 0.059; 0.062; 0.058 |

In general, HP should be proliferated during the co-cultivation with cells. However, in the experiment wherein HP was directly suspended in the cell culture medium DMEM and cultured in a cell incubator at 5% O₂, 37° C., it was found that the OD₆₂₀ value of the HP suspension decreased significantly over time rather than increase. A total of three OD₆₂₀ values were measured, which decreased gradually. HP culture solution incubated for 36 hours was directly spreaded on five Columbia media and cultured in a triple-gas incubator at 10% CO₂, 5% O₂, 85% N₂ and 37° C., it was found after five days that no colonies or HP growth were observed (see FIG. 6). The above results indicate that the inhibition of cell proliferation by HP during the co-cultivation with OMEC is due to the pathogenicity of HP itself, rather than insufficient cell nutrition caused by HP proliferation that consumes a large amount of nutrients.

### Example 7: Preparation of the Pharmaceutical Composition Comprising Scutellaria baicalensis and Cortex phellodendri as Well as the Effect Thereof Against HP

The pharmaceutical composition was prepared according to the method of Example 1, wherein the refined sesame oil, Scutellaria baicalensis and Cortex phellodendri (100 kg:5 kg:4 kg) were added to a reaction tank in step 1, and other steps were as same as in Example 1.

The pharmaceutical composition obtained in this example was subjected to the test method of Example 2, and the following results are obtained:

HP cultured in the Columbia medium, a specific medium for the culture of HP, grows normally, and the morphology, staining and biochemical reaction are all normal.

However, HP cultured in the Columbia medium containing different concentrations of the pharmaceutical composition of this example could not grow at all. HP could not grow even when the pharmaceutical composition is present in the lowest concentration of 5%.

The pharmaceutical composition obtained in this example was subjected to the test method of Example 3, and the following results are obtained:

On Day 3 of the cultivation of HP cultured in the Columbia medium free of the pharmaceutical composition, HP grows normally and forms an obvious lawn, the colonies are typical, and Gram stain shows that the bacteria has normal morphology, and has no variation. On Day 5 of the cultivation, HP cultured in the Columbia medium containing 0.3125% of the pharmaceutical composition of this example has variation obviously in morphology according to Gram stain. The effect of normal and variant HP culture on OMEC growth is observed through co-cultivation of OMEC with HP. On Day 4, 15 and 41 of the co-cultivation, no OMEC is observed under the microscope in 3 ml and 1 ml of normal HP suspension, indicating that OMEC are dead, and HP suspension has killed OMEC. On Day 4, 15 and 41 of the co-cultivation, OMEC could be observed under the microscope in 3 ml and 1 ml of variant HP suspension, indicating that OMEC are not killed by HP suspension. On Day 4 of the co-cultivation, normal OMEC could be observed under the microscope in the normal blank control well, and are health.

### Example 8: Preparation of the Pharmaceutical Composition Comprising Scutellaria baicalensis and Coptis chinensis as Well as the Effect Thereof Against HP

The pharmaceutical composition was prepared according to the method of Example 1, wherein the refined sesame oil, Scutellaria baicalensis and Coptis chinensis (100 kg:5 kg:4 kg) were added to a reaction tank in step 1, and other steps were as same as in Example 1.

The pharmaceutical composition obtained in this example was subjected to the test methods of Example 2 and 3. The results are similar to those of the composition obtained in Example 7, indicating that this composition can inhibit HP.

### Example 9: Preparation of the Pharmaceutical Composition Comprising Scutellaria baicalensis, Cortex phellodendri and Coptis chinensis as Well as the Effect Thereof Against HP

The pharmaceutical composition was prepared according to the method of Example 1, wherein the refined sesame oil, Scutellaria baicalensis and Coptis chinensis (100 kg:5 kg:5 kg:5 kg) were added to a reaction tank in step 1, and other steps were as same as in Example 1.

The pharmaceutical composition obtained in this example was subjected to the test methods of Example 2 and 3. The results are similar to those of the composition obtained in Example 7. However, HP cultured in the Columbia medium containing different concentrations of the pharmaceutical composition of this example could not grow at all. HP could not grow even when the pharmaceutical composition was present in the lowest concentration of 5%. With respect to HP cultured in the Columbia medium containing 0.3125% of the pharmaceutical composition of this example, on Day 4, 15 and 41 of the co-cultivation, no OMEC are observed under the microscope in 3 ml and 1 ml of normal HP suspension, indicating that all OMEC has been killed by HP suspension.

### Example 10: Preparation of the Pharmaceutical Composition Comprising Scutellaria baicalensis, Coptis chinensis, Cortex phellodendri, Pericarpium papaveris and Earthworm as Well as the Effect Thereof Against HP

The pharmaceutical composition was prepared according to the method of Example 1, wherein the refined sesame oil, Scutellaria baicalensis, Coptis chinensis, Cortex phellodendri, Pericarpium papaveris and earthworm (100 kg:5 kg:4 kg:4 kg:5 kg:5 kg) were added to a reaction tank in step 1, and other steps were as same as in Example 1.

The pharmaceutical composition obtained in this example was subjected to the test method of Example 2, and the following results are obtained:

HP cultured in the Columbia medium, a specific medium for the culture of HP, grows normally, and the morphology, staining and biochemical reaction are normal. However, HP cultured in the Columbia medium containing high and middle concentrations of the pharmaceutical composition of this example could not grow at all, while merely a few of HP cultured in the Columbia medium containing low concentrations of the pharmaceutical composition of this example could grow, indicating that the pharmaceutical composition of this example has a strong inhibition effect on HP.

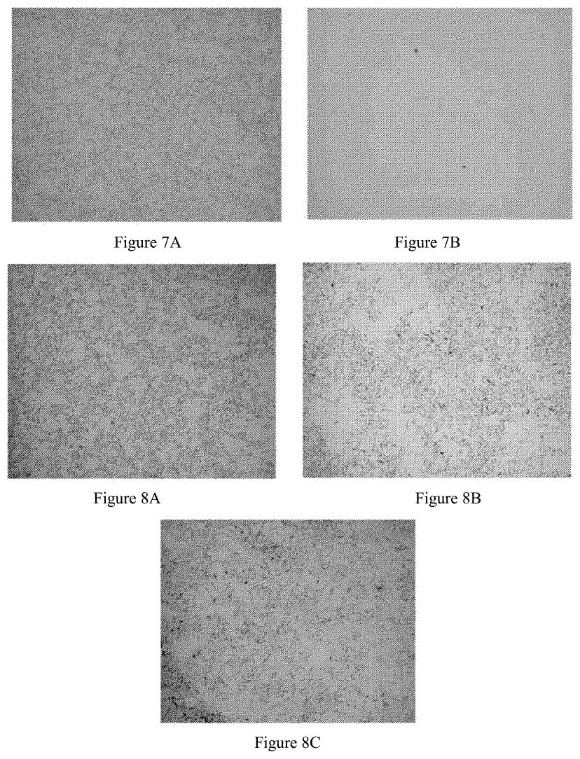

Specifically, HP cultured in Columbia medium grows normally, and the morphology is normal (see FIG. 7A). HP was cultured in a Columbia medium containing 10% of the pharmaceutical composition of this example for 72 hours, and the result shows that there is no bacterial growth in the medium. HP was cultured in a Columbia medium containing 5% of the pharmaceutical composition of this example for 72 hours, and the result shows that there is no bacterial growth in the medium. HP is cultured in a Columbia medium containing 2.5% of the pharmaceutical composition of this example for 72 hours, and the result shows that there is merely a few of bacterial grown in the medium (see FIG. 7B).

HP cultured in the Columbia medium, a specific medium for the culture of HP, grows normally, and the morphology, staining and biochemical reaction are normal. However, HP cultured in the Columbia medium containing 1.25% of the pharmaceutical composition of this example has variation obviously in morphology. The variation varies from common variation to obvious variation, from atypical variation to typical variation, and from insignificant variation to significant variation.

Specifically, HP cultured in Columbia medium grows normally, and the morphology is normal (see FIG. 8A). HP cultured in the Columbia medium containing 1.25% of the pharmaceutical composition of this example is in the early stage of variation, mainly manifesting as the cell body becomes longer (see FIG. 8B). HP cultured in the Columbia medium containing 1.25% of the pharmaceutical composition of this example is in the late stage of variation, mainly manifesting as the cell body becomes thinner and longer (see FIG. 8C which shows the dead HP).

The pharmaceutical composition obtained in this example was subjected to the test method of Example 3, and the following results are obtained: with respect to HP cultured in the Columbia medium containing as low as 0.3125% of the pharmaceutical composition of this example, on Day 4, 15 and 41 of the co-cultivation, no OMEC is observed under the microscope in 3 ml and 1 ml of normal HP suspension, indicating that all OMEC has been killed by HP suspension.

### Example 11: Effect of the Pharmaceutical Composition Against HP in Human Body

1. Materials and Methods

1.1 Clinical Data

22 patients with upper gastrointestinal ulcers and inflammation diagnosed by gastroscopic examination were enrolled (12 male, 10 female, age 29 to 71). Among them, 15 patients had single lesion, and other 7 patients had double or multiple lesions. There were 5 cases (sites) of gastric ulcer and duodenal ulcer respectively, 16 cases (sites) of chronic gastritis, 4 cases (sites) of gastric mucosal erosion, and 1 case (site) of esophageal ulcer inflammation.

1.2 Grouping and Treatment

(1) Grouping: According to voluntary principles, the patients were grouped into group A (15 cases), wherein the pharmaceutical composition prepared in Example 1 was administrated alone, and group B (7 cases), wherein the pharmaceutical composition prepared in Example 1 was administrated in combination with internal medicine routine treatment. The clinical symptoms, signs and results of gastroscopic examination after one month were observed to compare treatment efficacy.

(2) Administration method: Group A: with respect to esophagitis, gastric ulcer and inflammation patients, 2.5 g of the pharmaceutical composition prepared in Example 1 was administrated 4 times a day (half an hour before three meals and before going to bed), and the pharmaceutical composition was preferably administrated to a esophagitis patient through chew and swallow; with respect to duodenal ulcer patients, 4.0 g of the pharmaceutical composition prepared in Example 1 was administrated 4 times a day (half an hour before three meals and before going to bed); the total course of treatment was one month. Group B: the pharmaceutical composition prepared in Example 1 was administrated in the same way as in group A. Internal medicine routine treatment included oral administration of 20 mg of metoclopramide three times a day and 150 mg of ranitidine twice a day, or oral administration of 20 mg of Losec once a day for severe patients with hemorrhagic tendency. In order to accelerate the removal of HP infection, additional 500 mg of amoxicillin was administrated once a day for 7 days, or 0.5 g of clindamycin was administrated twice a day for 7 days. The course of treatment was one month.

(3) The symptoms and signs before the administration and corresponding changes and adverse reactions after the administration were recorded. An electronic gastroscopic examination was performed at the end of a course of treatment.

1.3 Criteria of Efficacy

(1) Cure: Symptoms and signs such as upper abdominal pain has been improved significantly and the black stool disappears after 10 days of administration. Symptoms and signs disappears after 20 days of administration. The mucosal inflammation disappears, and ulcer has been healed physiologically without obvious scars according to a gastroscopic examination performed after one month of administration. HP test turns negative.

(2) Improvement: Symptoms and signs such as upper abdominal pain has been improved slightly and the black stool disappears or has been relieved after 10 days of administration. Symptoms and signs such as upper abdominal pain has been improved significantly, the black stools disappears, and OB test turns negative or weak positive after 20 days of administration. The inflammation has been relieved, the ulcer area has been reduced by more than ½, or the ulcer has been healed with scars according to a gastroscopic examination performed after one month of administration. HP test turns from strong positive to negative or weak positive.

(3) Failure: Symptoms and signs have no obvious change after 20 days of administration. The inflammation has not been relieved, and the ulcer area has been reduced by less than ½ according to a gastroscopic examination performed after one month of administration. HP test is still positive.

2. Results

After 10 days of treatment, all the patients in both groups A and B feel that the symptoms have been improved, that is to say, the symptom improvement rate of both groups is 100%. After 20 days of treatment, the disappearance rate of symptom is 93.3% for group A, and 100% for group B. The gastroscopic examination performed after one month of treatment shows that the HP negative conversion rate is 53.3% (8/15) for group A, and 42.9% (3/7) for group B; the improvement rate is 40.0% (6/15) for group A, and 42.9% (3/7) for group B; and the failure rate is 6.7% (1/15) for group A, and 14.3% (1/7) for group B. The ulcer cure rate of groups A and B is 100%, wherein the rate of healing with scars is 6.7% for group A, and 14.3% for group B. The inflammatory lesion has been relieved significantly (100% in both groups A and B).

It can be seen that although group A is treated with the pharmaceutical composition prepared in Example 1 alone, it shows a similar result to that of group B which is treated with the pharmaceutical composition prepared in Example 1 in combination with chemical drugs (metoclopramide, ranitidine, amoxicillin and the like). The result demonstrates that the pharmaceutical composition of the present invention can replace the above chemical drugs to exert an therapeutic effect, including relieving clinical symptoms, protecting gastrointestinal mucosa, improving upper gastrointestinal ulcers, healing inflammation, controlling HP infection and the like. According to the result of the present study, the pharmaceutical composition of the present invention can rapidly alleviate symptoms such as the pain of an ulcers and inflammatory patient by preventing HP colonization or changing HP pathogenicity to prevent re-injury of injury factors and stimulation of nerve endings. The pharmaceutical composition improves local inflammation obviously, and provides a physiological environment and living substance for regenerative repair of the lesion sites. The pharmaceutical composition also facilities the physiologically regenerative repair of ulcers, and improves the quality of repair.

1. Use of a pharmaceutical composition in the preparation of medicaments of anti-HP, wherein the pharmaceutical composition is a pharmaceutical composition suitable for oral administration, comprising a homogenous mixture of edible oil, beeswax and β-sitosterol, wherein the beeswax in the composition forms microcrystals, the content of the beeswax is 0.5 to 50% and the content of the β-sitosterol is 0.1 to 20% by weight based on the total weight of the composition.

2. The use according to claim 1, wherein “anti-HP” refers to make the HP unable to grow and reproduction, make the HP reproduction slowly, make the HP variation, make the HP death and/or make reduction of the HP pathogenicity.

3. Use of a pharmaceutical composition in the preparation of medicaments for the treatment or prevention of diseases caused by HP, wherein the pharmaceutical composition is a pharmaceutical composition suitable for oral administration comprising a homogenous mixture of edible oil, beeswax and β-sitosterol, wherein the beeswax in the composition forms microcrystals, the content of the beeswax is 0.5 to 50% and the content of the β-sitosterol is 0.1 to 20% by weight based on the total weight of the composition.

4. The use according to claim 3, wherein the disease caused by HP comprises gastritis, gastric ulcer, duodenal ulcer, gastric cancer, gastric non-Hodgkin's lymphoma and gastric mucosa-associated lymphoid tissue lymphoma caused by HP infection.

5. The use according to claim 4, wherein the disease caused by HP is a disease caused in mammal, preferably human.

6. The use according to claim 1, characterized in that the content of the β-sitosterol in the pharmaceutical composition is 0.5 to 20% by weight.

7. The use according to claim 1, characterized in that the content of the β-sitosterol in the pharmaceutical composition is 1 to 10% by weight.

8. The use according to claim 1, characterized in that the content of the beeswax in the pharmaceutical composition is 3 to 30% by weight.

9. The use according to claim 1, characterized in that the content of the beeswax in the pharmaceutical composition is 5 to 20% by weight.

10. The use according to claim 1, characterized in that the content of the beeswax in the pharmaceutical composition is 6 to 10% by weight.

11. The use according to claim 1, characterized in that the edible oil in the pharmaceutical composition is corn oil, wheat germ oil, soybean oil, rice bran oil, rapeseed oil, sesame oil or fish oil.

12. The use according to claim 1, characterized in that the pharmaceutical composition further comprises propolis, and the content thereof is 0.1 to 30% by weight.

13. The use according to claim 1, characterized in that the pharmaceutical composition comprises water, and the content thereof is less than or equal to 1% by weight.

14. The use according to claim 1, characterized in that the dosage form of the oral pharmaceutical composition is selected from the group consisting of a tablet, pill, capsule, emulsion, gel, syrup and suspension.

15. The use according to claim 1, characterized in that the pharmaceutical composition further comprises Scutellaria baicalensis or Scutellaria baicalensis extract, and the content of Scutellaria baicalensis or Scutellaria baicalensis extract having 0.1 to 0.5% of baicalin is 2 to 5% by weight based on the total weight of the composition, the Scutellaria baicalensis is one or more Labiatae plants selected from the group consisting of Scutellaria viscidula bunge, Scutellaria amoena, Scutellaria rehderiana Diels, Scutellaria ikonnikovii Juz, Scutellaria likiangensis and Scutellaria hypericifolia.

16. The use according to claim 1, characterized in that the pharmaceutical composition further comprises Cortex phellodendri or Cortex phellodendri extract, and the content of Cortex phellodendri or Cortex phellodendri extract having 0.1 to 1% of obaculactone is 2 to 5% by weight based on the total weight of the composition, the Cortex phellodendri is selected from the group consisting of Phellodendron chinense Schneid, Phellodendron amurense, Phellodendron chinense Schneid var. omeiense, Phellodendron Schneid var. yunnanense and Phellodendron chinense Schneid var. falcutum.

17. The use according to claim 1, characterized in that the pharmaceutical composition further comprises 2 to 5% of Coptis chinensis or Coptis chinensis extract having 0.1 to 1% of berberine by weight based on the total weight of the composition.

18. The use according to claim 17, characterized in that the Scutellaria baicalensis extract is a Scutellaria baicalensis extract obtained in sesame oil, the Cortex phellodendri extract is a Cortex phellodendri extract obtained in sesame oil, and the Coptis chinensis extract is a Coptis chinensis extract obtained in sesame oil.

19. The use according to claim 1, characterized in that the pharmaceutical composition further comprises 2 to 5% of Scutellaria baicalensis or Scutellaria baicalensis extract having 0.1 to 0.5% of baicalin, 2 to 5% of Cortex phellodendri or Cortex phellodendri extract having 0.1 to 1% of obaculactone, 2 to 5% of Coptis chinensis or Coptis chinensis extract having 0.1 to 1% of berberine, 2 to 10% of Pericarpium papaveris or Pericarpium papaveris extract having 0.1 to 1% of narcotoline, and 2 to 10% of earthworm or earthworm extract by weight based on the total weight of the composition.

20. The use according to claim 1, characterized in that the pharmaceutical composition comprises 7% of beeswax, 1% of sterol, 0.5% of obaculactone, 0.3% of baicalin and 0.5% of berberine by weight based on the total weight of the composition.

21. The use according to claim 1, characterized in that the beeswax has microcrystals with a length of 0.1 to 100 microns.

22. The use according to claim 21, characterized in that at least two microcrystals of the beeswax in the pharmaceutical composition are polymerized into a microcrystal composite.

23. The use according to claim 22, characterized in that the microcrystals of the beeswax are sufficiently uniformly dispersed in the edible oil.
